(19) United States
(12) Patent Application Publication
(10) Pub. No.: US 2018/0036033 A1
(43) Pub. Date: Feb. 8, 2018

(54) SYSTEMS AND METHODS FOR ELECTRODE PLACEMENT IN DEEP MUSCLES AND NERVES USING ULTRASOUND GUIDANCE

(71) Applicants: Anthony R. IGNAGNI, Oberlin, OH (US); Raymond P. ONDERS, Shaker Heights, OH (US); Moustapha DIOP, Lorain, OH (US); Dan KELSCH, Fairview Park, OH (US); James E. GELBKE, North Royalton, OH (US)

(72) Inventors: Anthony R. IGNAGNI, Oberlin, OH (US); Raymond P. ONDERS, Shaker Heights, OH (US); Moustapha DIOP, Lorain, OH (US); Dan KELSCH, Fairview Park, OH (US); James E. GELBKE, North Royalton, OH (US)

(21) Appl. No.: 15/668,575

(22) Filed: Aug. 3, 2017

Related U.S. Application Data
(60) Provisional application No. 62/370,646, filed on Aug. 3, 2016.

(51) Int. Cl. A61B 17/34; A61N 1/05; A61B 8/12; A61B 6/03; A61B 5/04; A61B 5/0492

(52) U.S. Cl. CPC A61B 17/3403; A61B 5/04001; A61B 5/0492; A61B 17/3468; A61B 17/3423; A61B 8/12; A61B 6/032; A61N 1/05; A61B 2017/3413; A61B 2017/00929

(57) ABSTRACT

Systems and methods for implanting an electrode under ultrasound guidance in muscle, such as the diaphragm, or near nerve tissue can include inserting under ultrasound guidance a catheter or cannula into a body cavity of the patient proximate the target muscle or near the target nerve tissue; inserting under ultrasound guidance an insertion needle, with the electrode loaded into a lumen of the insertion needle, into the body cavity and into the target muscle or near the target nerve tissue; and withdrawing the insertion needle to expulse the electrode and lead from the central lumen of the insertion needle.

### CROSS REFERENCE TO RELATED APPLICATIONS

This application claims priority to U.S. Provisional Patent Application No. 62/370,646, filed Aug. 3, 2016, and titled “SYSTEMS AND METHODS FOR ELECTRODE PLACEMENT IN DEEP MUSCLES AND NERVES USING ULTRASOUND GUIDANCE,” which is herein incorporated by reference in its entirety.

This application may be related to U.S. Pat. No. 5,472,438; U.S. Pat. No. 5,797,923; U.S. Pat. No. 7,206,641; U.S. Pat. No. 9,050,005; U.S. Pat. No. 8,676,323; U.S. Pat. No. 7,962,215; U.S. Pat. No. 9,079,016; U.S. Pat. No. 8,478,412; U.S. Pat. No. 8,428,726; U.S. Pat. No. 7,840,270; and U.S. Patent Publication No. 2008/0287820 each of which is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

Embodiments of the invention relate generally to systems and methods for electrode placement within the body, and more specifically to systems and methods for electrode placement using ultrasound guidance.

### BACKGROUND

Diaphragm stimulation has been shown to be a beneficial therapy for treatment of a number of diseases and as an intervention to prevent additional morbidities during the course of other treatments, such as mechanical ventilation. To date, the placement of electrodes to stimulate the diaphragm has been done through surgical procedures, either intramuscular or direct nerve implants. Additional techniques have been developed to place stimulation electrodes transvenously or with natural orifice transluminal endoscopy. Those techniques introduce new risks that provide barriers for their clinical adoption.

Temporary recording electrodes have been placed in the diaphragm using ultrasound (U/S) guidance for placement.[1] The placement of these temporary electrodes are fine wire electrodes placed in costal diaphragm. Percutaneous electrodes have been placed in muscles for many years using percutaneous needles,[2-5] but those have been in superficial muscle or where placement does not have a risk of disrupting and/or damaging other organs or vessels. There is a need to target deep muscles, such as the diaphragm, or other deep tissues, such as nerve tissue, identify specific responsive locations, and place an electrode that may be retained for several days/weeks in that location. With surgical techniques developed to place intramuscular electrodes, this device and technique describes a system to place an electrode with local anesthetic at the patients' bedside.

This application describes systems, devices, and methods using a percutaneous approach to placement of stimulation electrodes without the concomitant complications of the existing techniques.

### SUMMARY OF THE DISCLOSURE

The present invention relates generally to systems and methods for electrode placement within the body, and more specifically to systems and methods for electrode placement in deep tissue using imaging guidance, such as ultrasound imaging, CT imaging, or imaging through a single camera.

In some embodiments, a system for placing an electrode in a muscle or in or near a nerve tissue of a patient using ultrasound or CT imaging is provided. The system includes an introducer needle with a lumen; a mapping stylet configured to be inserted through the lumen of the introducer needle, the mapping stylet having a proximal end and a distal end, the mapping stylet configured to deliver electrical stimulation to the muscle or nerve tissue; and an insertion needle having a lumen configured to receive the electrode, the insertion needle configured to be inserted into the lumen of the introducer needle.

In some embodiments, the system further includes a positioning cannula with a lumen, the positioning cannula configured to be inserted through the lumen of the introducer needle.

In some embodiments, the system further includes a stiffening stylet configured to be removably disposed in the introducer needle, the stiffening stylet configured to facilitate insertion of the introducer needle through the dermis, subcutaneous tissue, intercostal space, and/or muscle.

In some embodiments, the positioning cannula has a preformed bend and wherein the introducer needle is flexible and configured to conform to the preformed bend of the positioning cannula.

In some embodiments, the introducer needle includes a hub located at a proximal end of the introducer needle, the hub configured to facilitate pushing the introducer needle through the dermis, subcutaneous tissue, intercostal space, and/or muscle.

In some embodiments, the positioning cannula is coated with an electrically insulative coating.

In some embodiments, the positioning cannula has a proximal end that is free of the electrically insulative coating.

In some embodiments, the positioning cannula has an echogenic surface.

In some embodiments, the positioning cannula comprises a second lumen configured to allow a fluid to be injected through the positioning cannula.

In some embodiments, the positioning cannula has a proximal end with a notch configured to mate with a boss on the insertion needle such that a bevel at a distal end of the insertion needle is configured to enter the muscle or nerve tissue at an oblique angle.

In some embodiments, the positioning cannula further comprises a second lumen in fluid communication with an inflatable balloon located on a distal portion of the positioning cannula, wherein the inflatable balloon is configured to facilitate tangential orientation of the positioning cannula.

In some embodiments, the positioning cannula is shorter in length than the insertion needle, such that the insertion needle is configured to extend from the positioning cannula when the insertion needle is fully inserted into the positioning cannula.

In some embodiments, the positioning cannula has a depth scale on an outer surface of the positioning cannula, the depth scale configured to facilitate placement of the positioning cannula at a predetermined depth.

In some embodiments, the positioning cannula has an atraumatic tip.

In some embodiments, the introducer needle is configured to allow insertion of the insertion needle through dermal and other tissue or muscular layers without dislodging the electrode from the insertion needle.

In some embodiments, the positioning cannula has a distal portion that is bent to a predetermined angle.

In some embodiments, the mapping stylet is configured to prevent uptake of tissue or fluids into the positioning cannula during placement to the target muscle (diaphragm) or nerve.

In some embodiments, the system further includes a mapping device in communication with the mapping stylet.

In some embodiments, the mapping stylet comprises a removable collar to prevent extension from the positioning cannula until in the desired position.

In some embodiments, the distal end of the mapping stylet is echogenic.

In some embodiments, the mapping stylet is insulated from the proximal end towards the distal end, leaving a portion of the distal end deinsulated that corresponds to an exposed length of the electrode.

In some embodiments, the insertion needle is configured to be inserted through the lumen of the positioning cannula.

In some embodiments, the electrode has a distal tip with a deinsulated barb configured to hold the electrode it in place against a bevel of the insertion needle.

In some embodiments, the insertion needle is made of a flexible material and is configured to traverse a preformed bend along the positioning cannula.

In some embodiments, the insertion needle comprises a proximal end with a hub configured to facilitate manipulation of the insertion needle.

In some embodiments, the insertion needle comprises a proximal end and a distal end with a beveled tip and a boss proximate the proximal end of the insertion needle, the boss configured to align the insertion needle with the positioning cannula such that the beveled tip of the insertion needle is oriented with the muscle or near the nerve tissue at an oblique angle.

In some embodiments, the oblique angle is between 5 and 60 degrees.

In some embodiments, the boss on the insertion needle is configured to be seated in a mating notch on the positioning cannula such that the insertion needle is fully extended from the positioning cannula to a predetermined length.

In some embodiments, the insertion needle is longer in length than the positioning cannula such that a predetermined length of the insertion needle is configured to enter into the target muscle or near the nerve tissue.

In some embodiments, a method of placing an electrode in a target muscle or near a target nerve tissue of a patient is provided. The method includes inserting under imaging guidance a catheter or one or more cannulas into a body cavity of the patient toward the target muscle or target nerve tissue; inserting under imaging guidance an insertion needle, with the electrode loaded into a lumen of the insertion needle, through the catheter or one or more cannulas and into the body cavity and into the target muscle or near the target nerve tissue; and withdrawing the insertion needle to expulse the electrode and lead from the central lumen of the insertion needle, thereby deploying the electrode in the target muscle or near the target nerve tissue.

In some embodiments, the target muscle is the patient's diaphragm.

In some embodiments, the imaging guidance is ultrasound imaging.

In some embodiments, the imaging guidance is CT imaging.

In some embodiments, the method further includes introducing under imaging guidance a fluid through the catheter or one or more cannulas to create an effusion to separate the target muscle or target nerve tissue from the surrounding organs or tissue, thereby improving an imaging visibility of the target muscle or the target nerve tissue.

In some embodiments, the one or more cannulas is an introducer needle.

In some embodiments, the insertion needle has an echogenic tip.

In some embodiments, prior to inserting the insertion needle, the target muscle or target nerve tissue is tested by inserting a mapping stylet, under imaging guidance, through the the catheter or one or more cannulas into a body cavity of the patient proximate the target muscle or near the target nerve tissue; connecting a mapping device to the mapping stylet; stimulating the target muscle muscle or target nerve to generate a target response; verifying the target response under imaging observation; and withdrawing the mapping stylet.

In some embodiments, the method further includes verifying the electrode placement by delivering electrical stimulation through the electrode and identifying movement of the target muscle.

In some embodiments, the method further includes verifying the electrode placement by detecting electrical activity of the target muscle or target nerve tissue through the placed electrode.

In some embodiments, the method further includes verifying the electrode placement during a volitional contraction of the target muscle.

In some embodiments, the method further includes inserting a positioning cannula through the introducer needle towards the muscle or nerve tissue, and wherein the insertion needle is inserted through both the positioning cannula and the introducer needle.

In some embodiments, the method further includes aligning an alignment boss on the insertion needle with an alignment notch on the positioning cannula.

In some embodiments, the step of deploying the electrode comprises withdrawing the insertion needle while pressing forward or holding in position the positioning cannula.

In some embodiments, the step of deploying the electrode occurs during a stimulated contraction of the muscle at the target site.

In some embodiments, the method further includes delivering one or more stimulation pulses through the electrode to the target site to verify electrode placement.

In some embodiments, the method further includes detecting muscle contraction or nerve activity through the electrode.

In some embodiments, the step of detecting muscle contraction or nerve activity includes generating audible or visual feedback based on a magnitude of muscle contraction or nerve activity.

In some embodiments, the method further includes orienting the insertion needle at an oblique angle to the muscle or nerve tissue at the target site.

In some embodiments, the method further includes orienting the positioning cannula at an oblique angle to the muscle or nerve tissue at the target site.

In some embodiments, the positioning cannula is oriented at an oblique angle by inflating a balloon at a distal tip of the positioning cannula.

In some embodiments, the step of deploying the electrode includes using water pressure to eject the electrode from the insertion needle.

In some embodiments, the step of deploying the electrode includes using a guidewire to eject the electrode from the insertion needle.

In some embodiments, a method of placing an electrode in muscle or near nerve tissue of a patient is provided. The method includes inserting, under visualization from only a single laparoscopic camera, an introducer needle through a dermal layer and a subcutaneous tissue; inserting, under visualization from the single laparoscopic camera, an insertion needle through the introducer needle and into a target muscle or near a target nerve tissue, wherein an electrode is loaded into a lumen of the insertion needle; and deploying the electrode at the target muscle or near the target nerve tissue.

In some embodiments, the target muscle is the diaphragm.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 illustrates an embodiment of a system for inserting an electrode in deep tissue.

FIG. 2 illustrates another embodiment of a system for inserting an electrode in deep tissue.

FIG. 3 illustrates an embodiment of an electrode loaded in an insertion needle.

FIG. 4 illustrates an embodiment of an insertion needle with an alignment feature and a positioning cannula with a complementary alignment feature.

FIG. 5 illustrates an embodiment of the positioning cannula with an inflatable balloon for orienting the tip of the cannula.

FIG. 6 illustrates a stepped notch cannula that allows an insertion needle to be extended in controlled steps.

FIG. 7 illustrates a view under ultrasound imaging with effusion of the insertion needle and diaphragm.

FIGS. 8A-8D illustrate various CT imaging views of the diaphragm.

FIG. 9 illustrates the view under ultrasound imaging without effusion of the insertion needle and diaphragm.

### DETAILED DESCRIPTION

System and Device

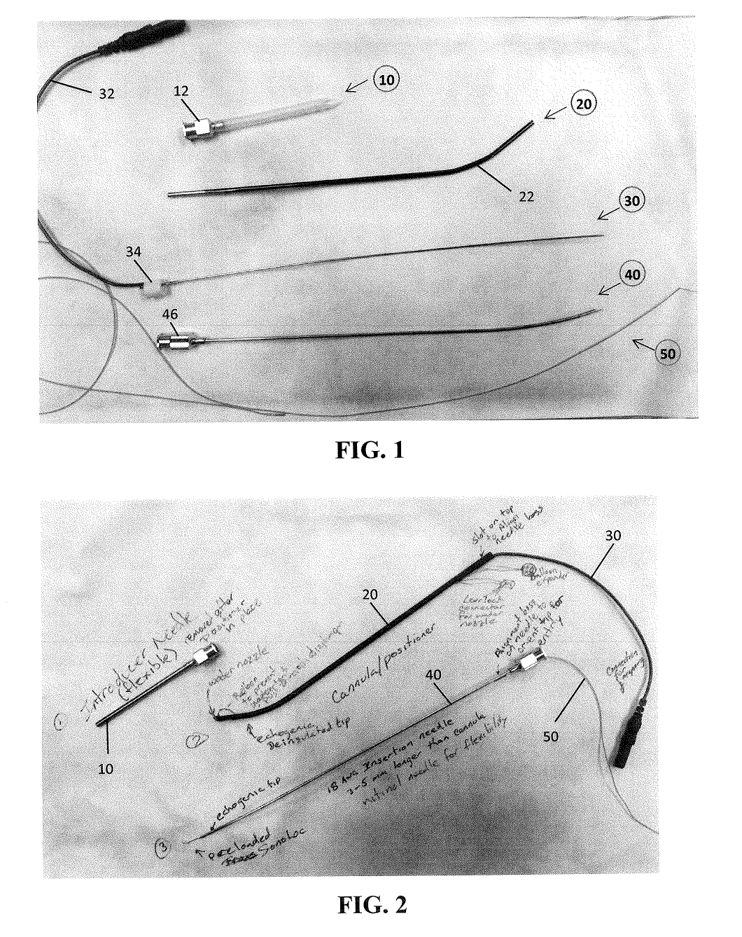

In some embodiments as shown in FIGS. 1-4, a system and device to place an electrode 50 in deep muscle, such as the diaphragm, or near deep nerve tissue, such as a phrenic nerve motor point, using ultrasound and/or auditory assisted signals, can include an introducer needle 10, a positioning cannula 20, a mapping stylet 30, and an insertion needle 40. In some embodiments, the use of positioning cannula 20 is optional, and instead, the insertion needle 40 with the electrode is placed directly through the introducer needle 10 under imaging.

In some embodiments, the introducer needle 10 may include a removable stiffening stylet to facilitate insertion through dermis, subcutaneous tissue, and other space, tissue, and/or muscle, such as the intercostal space. The depth of the insertion may be from about 0.78-4.91 cm, or about 0.5 to 5 cm, or about 0.25 to 10 cm.[6] Therefore, the length of the various needles, cannulas, stylets and/or catheters can be at least about 5 to 35 cm. A longer length allows an oblique approach to the target site. In some embodiments, the introducer needle 10, which may be removed after insertion of the positioning cannula 20 through the introducer needle 10, can be shorter than 5 to 35 cm.

In some embodiments, use of the introducer needle 10 would allow for the blunt tip positioning cannula 20 to enter the body cavity, such as a thoracic or abdominal cavity. The introducer needle 10 and/or positioning cannula 20 also allows the insertion needle 40 to pass through dermal and other tissue or muscular layers without dislodging the electrode 50 from the insertion needle 40 due to sheer drag forces on the electrode barb 52 that extends out of the distal tip of the insertion needle 40. Instead of inserting the insertion needle 40 directly through the dermal, tissue and/or muscular layers, the introducer needle 10 can be first inserted through the dermal, tissue, and/or muscular layers and the insertion needle 40 can then be inserted through the introducer needle and/or the positioning cannula 20. The introducer needle 10 may be flexible to allow for introduction of a pre-bent rigid positioning cannula 20. The introducer needle 10 may have an echogenic surface or marker, especially at the tip portion of the needle, to facilitate viewing of the introducer needle 10 under external ultrasound imaging.

In some embodiments, the introducer needle 10 may have a hub 12 or other fixture at the proximal end to facilitate pushing the needle through the dermis, subcutaneous tissue and other (intercostal) space/muscle. In some embodiments, the introducer needle 10 can have depth markings and/or alignment features.

In some embodiments, if a stiffening stylet is used, the stylet can be removed from the introducer needle 10 after insertion and then the positioning cannula 20 can be inserted into and through the introducer needle 10 until it is in the body cavity, such as the thoracic or abdominal cavity. The introducer needle 10 may then be optionally removed leaving the positioning cannula 20 in place. The positioning cannula 20 may be pre-bent to facilitate an oblique, near tangential orientation of the distal end of the positioning cannula to the tissue, muscle or near nerve tissue surface of the target site for electrode implantation. In some embodiments, the distal end of the positioning cannula may have a bend 22 that may be bent between about 5 to 70 degrees, or about 15 to 45 degrees, or about 50 to 70 degrees with respect to the longitudinal axis of the positioning cannula. In some embodiments, the insertion needle 40 is similarly bent as the positioning cannula 20. In some embodiments, the positioning cannula 20 can be flexible, or at least the bent portion 22 of the positioning cannula can be flexible, in order to allow the user to bend the positioning cannula as needed to an appropriate or desired angle, which may vary from patient to patient.

The surface of the lumen of the positioning cannula 20 may have an electrically insulative coating to insulate the positioning cannula 20 from the mapping stylet 30. The outer surface of the positioning cannula 20 may also be insulated. Alternatively, the mapping stylet 30 may be insulated up to the distal tip, which can be left uninsulated.

In some embodiments, the positioning cannula may also have an insulated outer surface and have an exposed or deinsulated surface area at the proximal end to allow a clip lead to be attached, and an exposed or desinsulated surface at the distal tip. This would allow the positioning cannula to be used as a mapping stylet or electrode and allow the user to stimulate the patient after insertion of the electrode at the diaphragm or other target site.

In some embodiments, the positioning cannula 20 may have an echogenic surface. For example, the surface can be covered or coated with an echogenic surface preparation or the surface can be etched to allow for heightened visibility with ultrasound. In some embodiments, the entire length of the cannula can be echogenic, while in other embodiments, only select portions of the cannula, such as the distal portion or end can be echogenic.

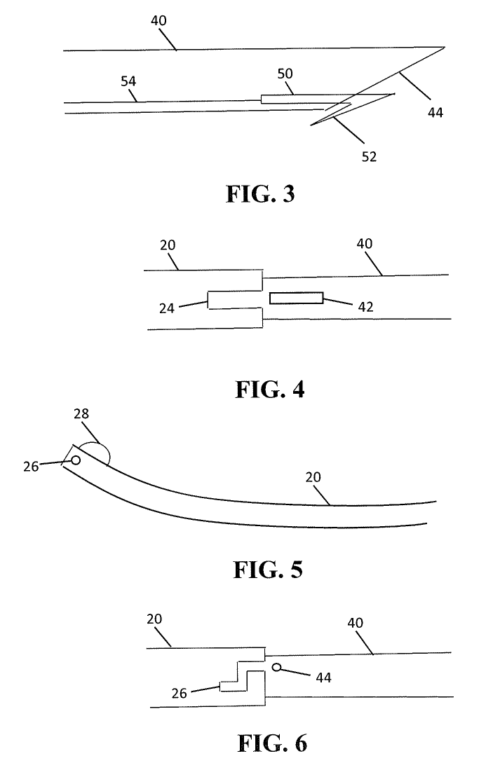

As shown in FIG. 5, the positioning cannula 20 may have a central lumen for receiving the mapping stylet and insertion needle and another lumen to allow sterile water or saline or another fluid to be injected from the proximal end to a flush port 26 the distal tip of the positioning cannula. This may allow a stream of water or saline or other fluid to facilitate expulsion of the electrode 50/electrode lead 54 from the insertion needle 40 shown in FIG. 3 by, for example, applying a small amount of pressure to the tissue interface at the tip of the cannula to apply pressure to the electrode barb 52. In some embodiments, the fluid can be injected through the central lumen of the positioning cannula 20 or directly through the injection needle 40.

In some embodiments as shown in FIG. 4, the positioning cannula 20 may have a notch 24, indent, slot or some other alignment feature at the proximal end to mate with a boss 42, bump, protuberance, or other complementary feature on the insertion needle 40. In some embodiments, the boss is less than about 0.25, 0.5, 0.75, or 1.0 cm in height. The alignment features facilitate orienting the insertion needle 40 with respect to the positioning cannula 20 and target site such that the insertion needle bevel 44 and electrode 50 carried by the insertion needle 40 enters the target site, such as muscle or near nerve tissue, at an appropriate oblique angle, which can be nearly tangential, in order to result in a shallow penetration depth for the needle and a shallow implantation depth for the electrode. For example, the insertion needle 40 and electrode 50 can enter the target site at less than 45, 30, or 15 degrees. In some embodiments, the needle bevel 44 can be oriented to face away from the tissue at the target site by aligning the boss 42 with the notch 24. This would also prevent or reduce the indentation of the muscle or nerve tissue and inadvertent deeper penetration of the insertion needle into the muscle or tissue.

In some embodiments as shown in FIG. 5, the positioning cannula 20 may have an inflation lumen from the proximal end to the distal end where an inflatable balloon 28 may be mounted to facilitate oblique, near tangential orientation of the distal end of the positioning cannula to prevent the cannula from indenting the target tissue and causing the insertion needle to penetrate the muscle or other tissue at the target site too deeply or at the wrong angle.

In some embodiments, the positioning cannula 20 can be shorter in length than the insertion needle 40, such that the insertion needle 40 is extended from the positioning cannula 20 when fully inserted into the positioning cannula 20. For example, the insertion needle 40 can be about 3 to 5 mm longer than the positioning cannula 20, which limits the depth of insertion of the insertion needle 40 and electrode 50. The positioning cannula 20 and insertion needle 40 may have a depth scale imprinted on the outer surface to facilitate placement at the appropriate depth. In other embodiments where the positioning cannula 20 is not used and the insertion needle 40 is inserted directly through the introducer 10, the introducer 10 and insertion needle 40 may have a depth scale imprinted on the outer surface to facilitate placement at the appropriate depth.

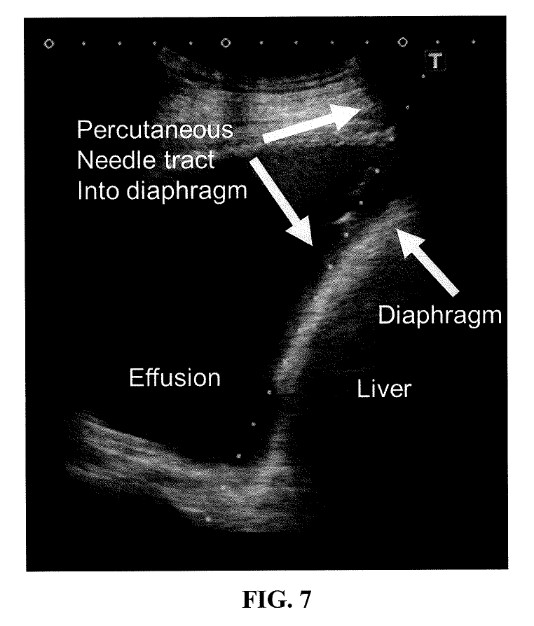

In addition, as shown in FIG. 6, the introducer 10 and/or positioning cannula 20 can have a stepped notch 26 or slot located at the proximal end of the instroducer 10 and/or positioning cannula 20 that mates with a mating feature 44, which can be a pin, a raised feature, or other complementary structure, on a proximal portion of the insertion needle 40 that allows the insertion needle 40 to be extended from the introducer 10 and/or positioning cannula 20 in two or more steps. The stepped notch 26 can have two or more steps to allow for the staged deployment of the insertion needle 40. In some embodiments, the tip of the insertion needle 40 can remain within the positioning cannula 20 or introducer 10 until the first step is engaged. In some embodiments, to extend or retract the insertion needle 40 the user can twist the introducer needle 10 relative to the positioning cannula 20 or introducer 10 such that the mating feature moves from one step to the next step, thereby allowing the insertion needle 40 to extend or retract from the positioning cannula 20 or introducer 10 in a controlled, stepwise fashion. The depth of insertion is controlled by the distance between the steps, which allows the insertion needle 40 to be extended out by a predetermined distance. For example, the notches can be spaced apart by about 1 cm to allow the insertion needle to be extended in 1 cm increments. This prevents or reduces the likelihood that the insertion needle 40 is overextended into and through the diaphragm.

A rubber diaphragm or seal may be placed over the proximal end of the introducer needle 40 to prevent escape of any artificial perfusion liquid or insufflation gas (if for example being placed under video laparoscopy or ultrasound) that may be used during the procedure. The diaphragm or seal can be made of other materials such as silicone or an elastomer.

The positioning cannula 20, introducer 10, and/or insertion needle 40 may have a knurled knob on the exterior portion of the proximal end to allow the surgeon to grasp the tool reliably. This is helpful because with the ultrasound gel and gloves, the tools can get slippery.

The notched end of the positioning cannula 20 or introducer 10 may be flared to help guide the insertion needle 40 into the cannula or introducer, rather than having to thread the insertion needle in.

In some embodiments, the mapping stylet 30 may be inserted through the central lumen of the positioning cannula 20 before the positioning cannula 20 is inserted into the body in order to prevent or reduce any coring of tissue or uptake of fluids into the positioning cannula 20 during insertion to the target site. The mapping stylet 30, which can be conductive and used to deliver electrical stimulation to the target site, also allows mapping of the tissues at and around the target site once the stylet is positioned at the target site, which can be muscle or near nerve tissue. Mapping the target site allows the user to identify the location of nerves and motor points, such as a phrenic nerve motor point, which can be a particularly advantageous location to place a stimulation electrode to efficiently facilitate muscle contraction. The mapping stylet 30 may include a cable 32 at the proximal end that can be connected to a mapping device which can generate the electrical stimulations to be delivered through the mapping stylet 32. The mapping stylet 32 may have a removable collar 34 to prevent extension from the positioning cannula until in the desired position. The mapping stylet 30 may have an echogenic surface preparation or etchings at the tip that becomes visible to ultrasound imaging after being extended from the positioning cannula 20. The mapping stylet 30 may be insulated from the proximal end toward the distal end, leaving a portion of the distal end deinsulated in order to deliver electrical stimulation to the target site. The length of the deinsulated portion may be approximately equal to the exposed length of the electrode.

In some embodiments, the insertion needle 40 may be inserted through the positioning cannula 20, after the mapping stylet 30 is removed. The insertion needle 40 has a central lumen that holds the electrode lead 54. The distal tip of the electrode 50 can have a deinsulated barb 52 that is bent back that holds the electrode 50 in place against the bevel 44 of the insertion needle 40, as shown in FIG. 3.

The insertion needle 40 may be made of flexible metal or other suitable material that can penetrate into the target muscle or near nerve tissue and traverse the positioning cannula 20, whether the positioning cannula 20 is straight of bent. If a straight positioning cannula is used, the insertion needle 40 may be made of a rigid metal or other rigid material. The insertion needle 40 may be coated with an echogenic material or etched, particularly the distal portion of the insertion needle 40 that extends past the distal end of the positioning cannula 20 when the insertion needle 40 is fully inserted into the positioning cannula 20. The insertion needle 40 may have a hub 46 at the proximal end to facilitate manipulation. As described above, the insertion needle 40 may have a boss 42 or other alignment feature near or at the proximal end of the insertion needle that facilitates alignment with a complementary alignment feature on the proximal end of the positioning cannula such that the needle bevel 44 is oriented with the muscle or other tissue at an appropriate oblique angle. The length of the insertion needle 40 is longer than the positioning cannula 20 such that the proper length of the insertion needle 40 will enter into the target muscle or other tissue without penetrating too far. In some embodiments, the length and/or location of the boss on the insertion needle is sufficient such that when fully seated in the mating notch 24 on the positioning cannula 20, the insertion needle 40 is fully extended from the positioning cannula 20. In some embodiments, the insertion needle 40 extends no more than 0.5, 1.0, 1.5, or 2.0 cm past the distal end of the positioning cannula 20 when fully extended. In some embodiments, depth markings can be provide on the boss 42 and/or notch or slot such that the user can determine how far the insertion needle 40 extends past the distal end of the positioning cannula 20 when the boss 42 is partially seated in the notch 24 or slot. After insertion of the insertion needle 40 and electrode 50 into the tissue at the target site, when the insertion needle 40 is withdrawn and extracted from the target site, the electrode 50 is expulsed due to the frictional shear force on the extended barb 52 of the electrode 50. In some embodiments, the electrode 50 or a portion of the electrode 50 can be coated with an echogenic coating or can be etched in order to enhance its visibility under ultrasound imaging.

Method

The system and devices described above can be used to percutaneously implant an electrode into deep muscle or near deep nerve tissue under ultrasound imaging guidance. Real-time external ultrasound imaging guidance allows the procedure to be performed more safely and efficiently at the patient's bedside. Certain deep tissues, such as the diaphragm, are visible under ultrasound. The regular, periodic movement of the diaphragm allows the diaphragm to be identified. In addition, a patient's breathing actions can be controlled by a mechanical ventilator, like causing an inspiration or holding breathing at the request of the physician, to assist with visualization.

In some embodiments, a catheter or cannula can be inserted into the body of a patient under ultrasound guidance until the distal end of the catheter or cannula approaches and nears the target site, which can be a deep muscle, such as the diaphragm, or can be near deep nerve tissue. The distal end of the catheter or cannula can be made echogenic as described above in order to enhance its visibility under ultrasound. The echogenic tip of the catheter or cannula can be guided to a visible anatomical landmark, such as the diaphragm.

In some embodiments, a fluid, such as water, saline, or an echogenic contrast fluid, such as a microbubble solution, can be injected through the catheter or cannula to create an artificial effusion, such as a pleural effusion, at and around the target site to separate the target muscle or tissue from the surrounding organs or tissue, thereby improving the ultrasound visibility of the target muscle or other tissue. In some embodiments, the patient is oriented upright or in a sitting position when the pleural effusion in introduced around the diaphragm. This orientation keeps the introduced fluid around the diaphragm, while in other orientations such as the prone position, the fluid may tend to flow away from the target site due to the effects of gravity. The orientation of the patient may be adjusted based on the target site in order to ensure that the effusion fluid remains around the target site. In some embodiments, ultrasound imaging can be used to confirm that the distal end of the positioning cannula is at an oblique, nearly tangential angle with respect to the target site. If needed, the angle of the positioning cannula can be adjusted based on the ultrasound imaging.

An electrode insertion needle can be inserted through the catheter or cannula to the target site. In some embodiments, an electrode can be preloaded into the insertion needle's central lumen before insertion through the catheter or cannula. The insertion needle can be inserted through the catheter or cannula into the body cavity and into the target site, which can be deep muscle, such as the diaphragm, or can be near other tissue, such as nerve tissue. As described above, the insertion needle can optionally have a surface finish near its distal tip to improve visibility under ultrasound. Ultrasound imaging can be used to control the depth and angle of insertion of the insertion needle into the tissue at the target site.

The insertion needle can be withdrawn to expulse the electrode and lead from the central lumen of the insertion needle. Ultrasound imaging can be used to confirm successful deployment of the electrode from the insertion needle. Optionally, the insertion needle can be withdrawn during a contraction of the muscle at the target site. The muscle contraction can either be volitional or evoked by applied stimulation. Volitional muscle contractions can also be performed during other steps of the procedure, such as insertion of the catheter or cannula and insertion of the insertion needle.

In some embodiments, a mapping device can be electrically connected to the electrode lead so that the electrode placement can be verified by delivering electrical stimulation through the electrode to evoke a muscle contraction, and visually or otherwise detecting muscle movement, such as through image analysis and/or motion detection. Alternatively, the mapping device can be used to detect native or evoked electrical activity from the muscle or nerve, or can detect other expected artifact activity, such as EKG activity or electrical activity from the heart, which can each be used to help confirm placement of the electrode.

In some embodiments, a mapping device can be electrically connected to a mapping stylet which can be inserted through the positioning cannula to map the area at and around the target site in order to determine a location for electrode placement. After the location is determined, the mapping stylet can be removed and the insertion needle can be inserted.

In some embodiments, an electrode can be placed in deep muscle or near deep nerve tissue using a similar approach that also involves ultrasound imaging guidance. An introducer needle with an echogenic finish can be inserted through the dermal layer, subcutaneous tissue and other intercostal space/muscle/tissue under ultrasound imaging guidance. In some embodiments, a stiffening stylet can be placed into the introducer needle before the introducer needle is inserted in order to reduce tissue coring and/or fluid uptake. After the introducer needle has been inserted, the stiffening stylet, if used, can be removed. A positioning cannula with an echogenic finish, with a mapping stylet inserted in the positioning cannula's central lumen, can be inserted through the introducer needle under ultrasound imaging guidance until the target site or anatomical landmark that is visible under ultrasound imaging is approached. As above, a fluid may be injected through the positioning cannula to separate the target site from the surrounding tissues, thereby increasing the target site's visibility under ultrasound. As above, the angle of the positioning cannula to the tissue at the target site can be determined and adjusted under ultrasound imaging to ensure that the angle is oblique and can be nearly tangential.

In some embodiments, the mapping stylet can have a collar that can serve as a stop to restrict insertion of the mapping stylet through the positioning cannula. For example, the stop can be used during the insertion of the positioning cannula to keep the mapping stylet within the positioning cannula, and then the collar can be removed to allow the mapping stylet to be extended out of the positioning cannula to map the tissue at the target sit. In some embodiments, removing the collar from the mapping stylet allows the mapping stylet to be extended from the positioning cannula by about 4-5 mm, or up to 5, 10, or 15 mm. The mapping stylet can have an echogenic finish and can also be visualized under ultrasound imaging with reference to anatomical landmarks at the target site. A mapping device can be electrically connected to the mapping stylet so that electrical stimulation pulses can be delivered to the tissue at the target site through a deinsulated tip of the mapping stylet. The stimulation pulses can cause muscle at the target site to contract, which can be visually detected or detected using the mapping device through image analysis and/or motion detection. Alternatively, the mapping device can detect muscle or nerve electrical activity, which can be evoked and/or native, and can provide audible feedback based on the amount of the muscle or nerve activity detected.

The mapping stylet can be positioned using the feedback to gain the optimal or greatest response to the mapping of the target site, thereby identifying the implantation site for the electrode. Once the implantation site is identified with the mapping, the mapping stylet can be withdrawn from the positioning cannula while holding the positioning cannula in place. While maintaining the positioning cannula in place, inserting the insertion needle with an electrode into the central lumen of the positioning cannula.

In some embodiments, the insertion needle can have an alignment boss, protrusion or feature on a proximal portion of the insertion needle that can be aligned with a complementary alignment notch, groove, slot or feature on a proximal portion of the positioning cannula. Aligning the two complementary alignment features ensures that the bevel of the insertion needle is oriented in the proper direction when it exits the positioning cannula. The insertion needle can be fully inserted by sliding the boss fully into the notch, thereby extending the insertion needle into the deep muscle or near deep nerve tissue. Ultrasound imaging can be used to monitor and control the angle and depth of insertion of the insertion needle into the tissue.

The insertion needle can be withdrawn from the positioning cannula to deploy the electrode. Optionally, the insertion needle can be removed during either a volitional or evoked contraction of the muscle at the target site. The insertion needle can be withdrawn while pressing forward with the positioning cannula and/or keeping the positioning cannula in place in order to expulse the electrode and lead from the central lumen of the insertion needle. The insertion needle and the positioning cannula can be removed from the patient's body leaving the electrode in place. Placement of the electrode can be confirmed using ultrasound imaging.

As described above, a mapping device can be electrically connected to the electrode lead and electrode placement can be verified by delivering electrical stimulation through the electrode and visually identifying and/or detecting using image and/or motion processing muscle muscle movement. Alternatively, the mapping device can detect electrical activity, evoked or natural, from the muscle, nerve, or other expected artifact (EKG) activity.

Optionally, as described above, a fluid can be injected through the positioning cannula to create an artificial effusion between the muscle or other tissue at the target site and surrounding organs and tissue, to both improve ultrasound imaging at the target site and to create space to avoid potential inadvertent puncture of surrounding organs/tissue. This can be done after insertion of the positioning cannula and before insertion of the insertion needle into the tissue.

Optionally, a balloon at the distal tip of the positioning cannula can be inflated, or a wire or other mechanical structure can be extended at the distal end to orient the positioning cannula in an oblique, nearly tangential angle to the muscle or other tissue at the target site.

Optionally, water, saline or another fluid can be sprayed through a lumen of the positioning cannula to apply pressure to the electrode tip during withdrawal of the insertion needle to facilitate expulsion of the lead and electrode from the insertion needle.

Optionally, a guidewire can be inserted into the lumen of the insertion needle to help push the electrode out of the insertion needle.

In some embodiments, external ultrasound imaging can be used to examine the implantation of the electrode and determine whether the implantation was successful, or whether the electrode should be reimplanted. For example, hematoma formation and other injuries at the implantation site may be detected using ultrasound imaging.

FIG. 7 illustrates a view under ultrasound imaging of a percutaneous needle tract into the diaphragm with effusion to enhance imaging.

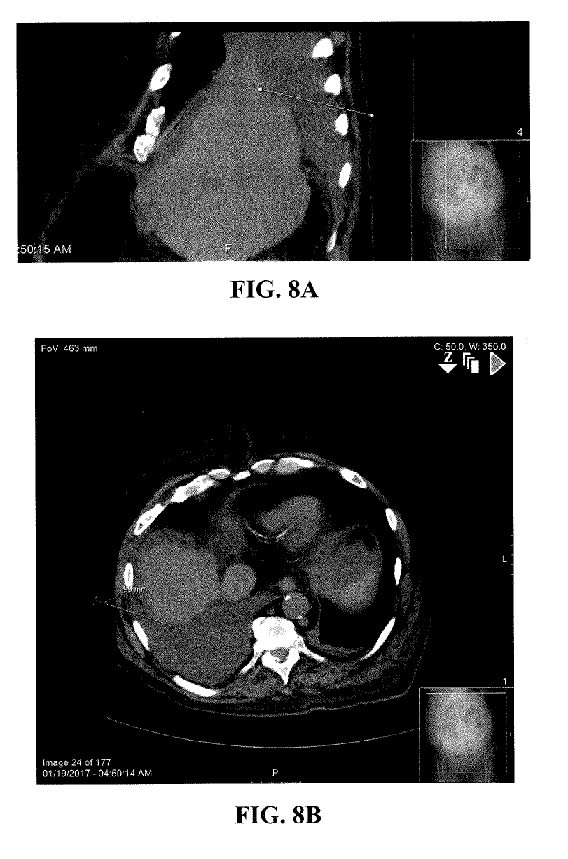

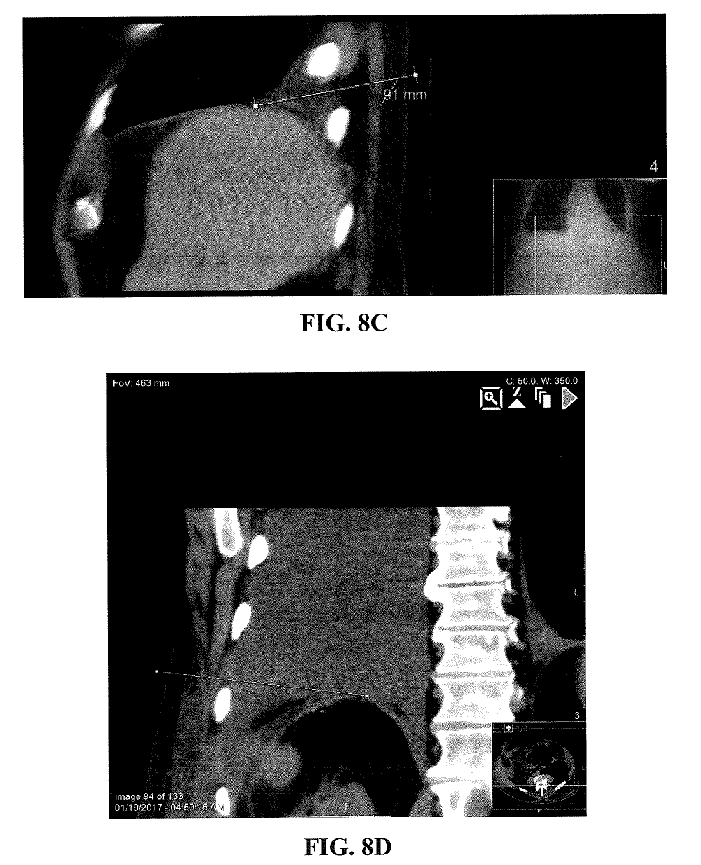

Although the system and methods described herein have been described with reference to ultrasound imagining, these systems and methods can be adapted to other imaging modalities such as computed tomography (CT) imaging. All or many of the tools described herein can be used in CT imaging with little or no alterations. If desired, to enhance contrast the devices can be provided with radiopaque markings in place of the echogenic markings above. FIGS. 8A-8D illustrate various CT imaging views of the diaphragm. FIG. 8A is a CT showing right diaphragm access point through effusion. FIG. 8B is another view of the same patient shown in FIG. 8A showing access. FIG. 8C is another CT with smaller effusion with access to the medial diaphragm. FIG. 8D is a CT showing large posterior effusion with access point.

### EXAMPLE 1

A first pig was used for exploratory procedures. Initial placement of laparoscopic trocars and view of diaphragm was from the abdominal aspect. The diaphragm was stimulated using a dissector, and diaphragm contraction was able to be visualized on ultrasound. An intentional pleural effusion was created by placing liquid in thoracic cavity. When the introducer needle was placed in thoracic cavity, some of the pleural effusion was leaked from the introducer needle.

In this case, the view of diaphragm was not substantially improved with the introduction of the pleural effusion. This was possibly due to the orientation of the pig on the table, e.g. gravity was not placing introduced effusion against diaphragm as it would in a sitting patient. Electrodes were placed into diaphragm using insertion needle, under U/S view as well as video laparoscopic view.

Since the pig was first insufflated for laparoscopic view, it seems that this may have made the U/S view more difficult. It also could have been the learning curve of the unfamiliar Philips U/S machine or accommodation of viewing U/S images.

The artificial pleural effusion in the pig model did not seem to work. This may have been due to the orientation of the pig, relative to gravity and diaphragm position, or due to the leak of liquid from the introducer needle.

There was difficulty in manipulating the introducer needle, cannula, insertion needle, and U/S probe all at once. This was partially due to the lack of or insufficient size of handling hubs on each of the tool components. It was also made more difficult by the insufficient length of the tools that were made for this experiment.

### EXAMPLE 2

Another experiment was performed using a second pig to demonstrate that the system and method can be used to identify the pig's diaphragm using an ultrasound (U/S) probe and then guide placement of an electrode into the diaphragm.

The introducer needle was inserted into the thorax and placed in position on the thoracic side of diaphragm (verified with U/S) through the dermal, subcutaneous, and muscular layers. The mapping stylet was then inserted into the introducer needle, and the diaphragm was stimulated using the mapping stylet to assure approximate positioning (contraction viewed with U/S). The mapping stylet was then removed and the insertion needle, loaded with an electrode, was inserted into the introducer needle. The electrode was inserted into the muscle and the surgeon was able to feel the force transient as the needle entered into the diaphragm muscle. The insertion needle could be viewed on the U/S. The insertion needle was partially withdrawn and the muscle stimulated and contraction was viewed and confirmed under U/S visualization. Once proper placement of the electrode was verified, the insertion needle was extracted from the body, leaving the electrode in placed in the diaphragm, and then the insertion needle was removed from the lead of the electrode. The introducer needle was then extracted from the body and then removed from the lead of the electrode. The electrode was again tested by stimulating the diaphragm and the resulting contraction was viewed with U/S. These steps were repeated for each electrode that was inserted into the diaphragm. Note that in this example, a positioning cannula was not used, and instead, the insertion needle with electrode was inserted directly through the introducer needle.

In addition, an abdominal side placement was performed on the second pig to provide laparoscopic visualization as a comparison with U/S visualization. The laparoscopic visualization was done with only a single camera.

Laparoscopic incisions were made and trocars placed at the umbilicus for the video camera and laterally for disposable electrode delivery instruments, such as those described in U.S. Pat. No. 5,797,923, which is hereby incorporated by reference in its entirety. The disposable instrument, with electrode, was inserted through a trocar and into the abdomen towards the diaphragm. The electrode was inserted into the diaphragm muscle under video observation using a single laparoscopic camera. Once placed into the diaphragm, the instrument was extracted from the body and removed from the lead, leaving the electrode in place in the diaphragm. The lead was then inserted fully into the abdomen for later retrieval through a common exit site. These steps were repeated for each electrode that was implanted. After all electrodes were placed in the diaphragm, the leads were brought out through a trocar and the trocar removed from the leads. The right and left side leads were then stimulated individually to identify which external ends corresponded to which side.

Mapping instruments as described above or in U.S. Pat. Nos. 5,472,438 and 7,206,641, which are herein incorporated by reference in their entireties, can be used to identify phrenic nerve motor points that can serve as implantation sites for the electrodes.

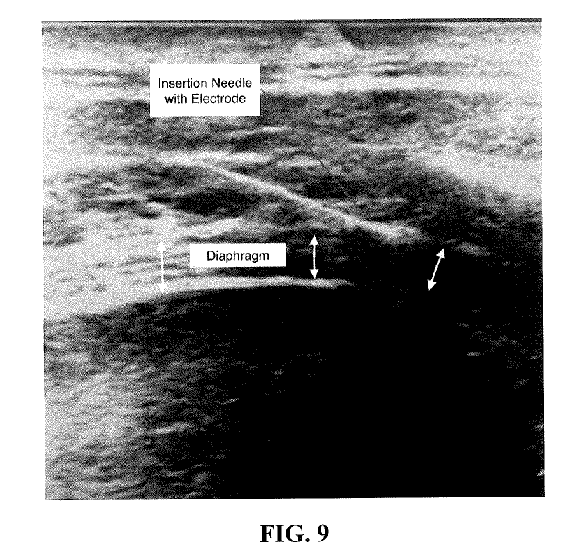

Under U/S guided placement with no artificial perfusion, instruments were clearly seen on U/S and diaphragm was clearly visualized, as shown in FIG. 9. Mapping stimulation was useful to see contraction and verify that the instruments were at the diaphragm. The positioning cannula was not used on placement of the mapping stylet or the insertion needle on the second pig. Good contractions of both right and left diaphragms were observed with U/S for all placements of electrodes. One electrode on the pig's right side did elicit some intercostal muscle recruitment. Both surgeons said they could feel the force change on the needle as they inserted into the muscle tissue.

Laparoscopic trocars were then place and inferior aspect of diaphragm was viewed for any electrodes protruding through diaphragm or damage to organs in the abdominal cavity. One electrode was seen to be through the diaphragm 2-3 cm. No evidence of damage to abdominal organs was seen. The protruding electrode was pulled into the diaphragm, with traction on the external portion of the lead. Placement of the electrodes with the disposable implant instrument was uneventful.

Tidal volumes and pressures were attempted to be recorded, but no volumes were seen on the ventilator, in spite of seeing vigorous contractions of the diaphragm with stimulation. Therefore, no additional data was collected on this pig. Visual observation showed all electrodes elicited good contractions, with the additional recruitment of the intercostals with the one U/S guidance placed electrode. Very similar contractions were observed by both sets of electrodes (U/S and laparoscopically placed). All electrodes were removed, fully intact, at the end of the experiment with gentle traction.

### EXAMPLE 3

In a third pig, the diaphragm was identified with an ultrasound (U/S) probe using a modified procedure that included the use of a positioning cannula. First, the introducer needle was placed in position on the thoracic side of diaphragm (verified with U/S) through the dermal, subcutaneous, and muscular layers. The positioning cannula was introduced into the introducer needle and advanced to the diaphragm (verified with U/S), and the introducer needle was left in place. The mapping stylet was then inserted into the positioning cannula and the diaphragm stimulated to assure approximate positioning (contraction viewed with U/S). The mapping stylet was then removed from the positioning cannula, and the insertion needle, with electrode, was inserted through the positioning cannula and into the diaphragm. The electrode was inserted into the muscle along with the insertion needle and the surgeon was able to feel the force transient as the needle entered into the diaphragm muscle. The insertion needle was viewed under U/S as it was inserted into the diaphragm. The insertion needle was partially withdrawn, leaving behind the electrode, and the muscle stimulated and contraction viewed with U/S to verify electrode placement. Once electrode placement was verified, the positioning cannula and insertion needle were extracted together from the body and removed from the electrode lead. The introducer needle was then extracted from the body and then removed from the electrode lead. The electrode was again tested by delivering electrical stimulation to the diaphragm and contraction was viewed with U/S. These steps were repeated for each electrode that was inserted.

An abdominal side placement was also performed on the third pig to provide laparoscopic visualization as a comparison with U/S visualization.

A single laparoscopic incision was made at the umbilicus, a trocar was inserted through the incision, and a video camera introduced through that port. The introducer needle was placed in position on the abdominal side of the diaphragm (verified with video) through the dermal, subcutaneous, and muscular layers. The insertion needle, with an electrode loaded, was inserted into the introducer needle, and advanced to the diaphragm under video observation. The insertion needle and electrode was inserted into the muscle under video observation. Once placed into the diaphragm, the insertion needle was extracted from the body, leaving the electrode in place in the diaphragm, and the insertion needle was removed from the electrode lead. The introducer needle was then extracted from the body and then removed from the electrode lead. These steps were repeated for each electrode.

Under U/S guided placement with no artificial perfusion, the positioning cannula was used on placement of mapping stylet and insertion needle for the third pig. Good contractions of both right and left diaphragms were observed with U/S for all placements of electrodes. Tidal volume with all four electrodes was measured following the placement with the U/S guidance.

A single laparoscopic video port was used to observe the insertion of electrodes using the insertion needle rather than disposable implant instruments. This technique was found to be very easy and quick, with four electrodes attempted and successfully implanted. Laparoscopic insufflation was taken down and the tidal volumes checked. Measurements of tidal volumes showed that U/S guidance and a single laparoscopic camera were able to successfully guide implantation of electrodes in the diaphragm.

It is understood that this disclosure, in many respects, is only illustrative of the numerous alternative device embodiments of the present invention. Changes may be made in the details, particularly in matters of shape, size, material and arrangement of various device components without exceeding the scope of the various embodiments of the invention. Those skilled in the art will appreciate that the exemplary embodiments and descriptions thereof are merely illustrative of the invention as a whole. While several principles of the invention are made clear in the exemplary embodiments described above, those skilled in the art will appreciate that modifications of the structure, arrangement, proportions, elements, materials and methods of use, may be utilized in the practice of the invention, and otherwise, which are particularly adapted to specific environments and operative requirements without departing from the scope of the invention. In addition, while certain features and elements have been described in connection with particular embodiments, those skilled in the art will appreciate that those features and elements can be combined with the other embodiments disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”. “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed. then 11, 12, 13. and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

### REFERENCES

1. Boon, A. J., et al., Ultrasound-guided needle EMG of the diaphragm: technique description and case report. Muscle Nerve, 2008. 38(6): p. 1623-6.
2. Akers, J. M., et al., Tissue response to chronically stimulated implanted epimysial and intramuscular electrodes. IEEE Trans Rehabil Eng, 1997. 5(2): p. 207-20.
3. Caldwell, C. W. and J. B. Reswick, A percutaneous wire electrode for chronic research use. IEEE Trans Biomed Eng, 1975. 22(5): p. 429-32.
4. Chae, J., D. Yu, and M. Walker, Percutaneous, intramuscular neuromuscular electrical stimulation for the treatment of shoulder subluxation and pain in chronic hemiplegia: a case report. Am J Phys Med Rehabil, 2001. 80(4): p. 296-301.
5. Marsolais, E. B. and R. Kobetic, Implantation techniques and experience with percutaneous intramuscular electrodes in the lower extremities. J Rehabil Res Dev, 1986. 23(3): p. 1-8.
6. Shahgholi, L., et al., Diaphragm depth in normal subjects. Muscle Nerve, 2014. 49(5): p. 666-8.

What is claimed is:

1. A system for placing an electrode in a muscle or in or near a nerve tissue of a patient using ultrasound or CT imaging, the system comprising:
an introducer needle with a lumen;
a mapping stylet configured to be inserted through the lumen of the introducer needle, the mapping stylet having a proximal end and a distal end, the mapping stylet configured to deliver electrical stimulation to the muscle or nerve tissue; and
an insertion needle having a lumen configured to receive the electrode, the insertion needle configured to be inserted into the lumen of the introducer needle.

2. The system of claim 1, further comprising a positioning cannula with a lumen, the positioning cannula configured to be inserted through the lumen of the introducer needle.

3. The system of claim 1, further comprising a stiffening stylet configured to be removably disposed in the introducer needle, the stiffening stylet configured to facilitate insertion of the introducer needle through the dermis, subcutaneous tissue, intercostal space, and/or muscle.

4. The system of claim 2, wherein the positioning cannula has a preformed bend and wherein the introducer needle is flexible and configured to conform to the preformed bend of the positioning cannula.

5. The system of claim 1, wherein the introducer needle includes a hub located at a proximal end of the introducer needle, the hub configured to facilitate pushing the introducer needle through the dermis, subcutaneous tissue, intercostal space, and/or muscle.

6. The system of claim 2, wherein the positioning cannula is coated with an electrically insulative coating.

7. The system of claim 6, wherein the positioning cannula has a proximal end that is free of the electrically insulative coating.

8. The system of claim 2, wherein the positioning cannula has an echogenic surface.

9. The system of claim 2, wherein the positioning cannula comprises a second lumen configured to allow a fluid to be injected through the positioning cannula.

10. The system of claim 2, wherein the positioning cannula has a proximal end with a notch configured to mate with a boss on the insertion needle such that a bevel at a distal end of the insertion needle is configured to enter the muscle or nerve tissue at an oblique angle.

11. The system of claim 2, wherein the positioning cannula further comprises a second lumen in fluid communication with an inflatable balloon located on a distal portion of the positioning cannula, wherein the inflatable balloon is configured to facilitate tangential orientation of the positioning cannula.

12. The system of claim 2, wherein the positioning cannula is shorter in length than the insertion needle, such that the insertion needle is configured to extend from the positioning cannula when the insertion needle is fully inserted into the positioning cannula.

13. The system of claim 2, wherein the positioning cannula has a depth scale on an outer surface of the positioning cannula, the depth scale configured to facilitate placement of the positioning cannula at a predetermined depth.

14. The system of claim 2, wherein the positioning cannula has an atraumatic tip.

15. The system of claim 1, wherein the introducer needle is configured to allow insertion of the insertion needle through dermal and other tissue or muscular layers without dislodging the electrode from the insertion needle.

16. The system of claim 2, wherein the positioning cannula has a distal portion that is bent to a predetermined angle.

17. The system of claim 2, wherein the mapping stylet is configured to prevent uptake of tissue or fluids into the positioning cannula during placement to the target muscle (diaphragm) or nerve.

18. The system of claim 1, further comprising a mapping device in communication with the mapping stylet.

19. The system of claim 2, wherein the mapping stylet comprises a removable collar to prevent extension from the positioning cannula until in the desired position.

20. The system of claim 1, wherein the distal end of the mapping stylet is echogenic.

21. The system of claim 1, wherein the mapping stylet is insulated from the proximal end towards the distal end, leaving a portion of the distal end deinsulated that corresponds to an exposed length of the electrode.

22. The system of claim 2, wherein the insertion needle is configured to be inserted through the lumen of the positioning cannula.

23. The system of claim 1, wherein the electrode has a distal tip with a deinsulated barb configured to hold the electrode it in place against a bevel of the insertion needle.

24. The system of claim 2, wherein the insertion needle is made of a flexible material and is configured to traverse a preformed bend along the positioning cannula.

25. The system of claim 1, wherein the insertion needle comprises a proximal end with a hub configured to facilitate manipulation of the insertion needle.

26. The system of claim 2, wherein the insertion needle comprises a proximal end and a distal end with a beveled tip and a boss proximate the proximal end of the insertion needle, the boss configured to align the insertion needle with the positioning cannula such that the beveled tip of the insertion needle is oriented with the muscle or near the nerve tissue at an oblique angle.

27. The system of claim 26, wherein the oblique angle is between 5 and 60 degrees.

28. The system of claim 26, wherein the boss on the insertion needle is configured to be seated in a mating notch on the positioning cannula such that the insertion needle is fully extended from the positioning cannula to a predetermined length.

29. The system of claim 2, wherein the insertion needle is longer in length than the positioning cannula such that a predetermined length of the insertion needle is configured to enter into the target muscle or near the nerve tissue.

30. A method of placing an electrode in a target muscle or near a target nerve tissue of a patient, the method comprising:
inserting under imaging guidance a catheter or one or more cannulas into a body cavity of the patient toward the target muscle or target nerve tissue;
inserting under imaging guidance an insertion needle, with the electrode loaded into a lumen of the insertion needle, through the catheter or one or more cannulas and into the body cavity and into the target muscle or near the target nerve tissue; and
withdrawing the insertion needle to expulse the electrode and lead from the central lumen of the insertion needle, thereby deploying the electrode in the target muscle or near the target nerve tissue.

31. The method of claim 30, wherein the target muscle is the patient's diaphragm.

32. The method of claim 30, wherein the imaging guidance is ultrasound imaging.

33. The method of claim 30, wherein the imaging guidance is CT imaging.

34. The method of claim 30, further comprising introducing under imaging guidance a fluid through the catheter or one or more cannulas to create an effusion to separate the target muscle or target nerve tissue from the surrounding organs or tissue, thereby improving an imaging visibility of the target muscle or the target nerve tissue.

35. The method of claim 30, wherein the one or more cannulas is an introducer needle.

36. The method of claim 30, wherein the insertion needle has an echogenic tip.

37. The method of claim 30, wherein prior to inserting the insertion needle, the target muscle or target nerve tissue is tested by:
inserting a mapping stylet, under imaging guidance, through the the catheter or one or more cannulas into a body cavity of the patient proximate the target muscle or near the target nerve tissue;
connecting a mapping device to the mapping stylet;
stimulating the target muscle muscle or target nerve to generate a target response;
verifying the target response under imaging observation; and
withdrawing the mapping stylet.

38. The method of claim 30, further comprising verifying the electrode placement by delivering electrical stimulation through the electrode and identifying movement of the target muscle.

39. The method of claim 30, further comprising verifying the electrode placement by detecting electrical activity of the target muscle or target nerve tissue through the placed electrode.

40. The method of claim 39, further comprising verifying the electrode placement during a volitional contraction of the target muscle.

41. The method of claim 35, further comprising inserting a positioning cannula through the introducer needle towards the muscle or nerve tissue, and wherein the insertion needle is inserted through both the positioning cannula and the introducer needle.

42. The method of claim 41, further comprising aligning an alignment boss on the insertion needle with an alignment notch on the positioning cannula.

43. The method of claim 41, wherein the step of deploying the electrode comprises withdrawing the insertion needle while pressing forward or holding in position the positioning cannula.

44. The method of claim 30, wherein the step of deploying the electrode occurs during a stimulated contraction of the muscle at the target site.

45. The method of claim 30, further comprising delivering one or more stimulation pulses through the electrode to the target site to verify electrode placement.

46. The method of claim 30, further comprising detecting muscle contraction or nerve activity through the electrode.

47. The method of claim 30, wherein the step of detecting muscle contraction or nerve activity comprises generating audible or visual feedback based on a magnitude of muscle contraction or nerve activity.

48. The method of claim 30, further comprising orienting the insertion needle at an oblique angle to the muscle or nerve tissue at the target site.

49. The method of claim 35, further comprising orienting the positioning cannula at an oblique angle to the muscle or nerve tissue at the target site.

50. The method of claim 49, wherein the positioning cannula is oriented at an oblique angle by inflating a balloon at a distal tip of the positioning cannula.

51. The method of claim 30, wherein the step of deploying the electrode comprises using water pressure to eject the electrode from the insertion needle.

52. The method of claim 30, wherein the step of deploying the electrode comprises using a guidewire to eject the electrode from the insertion needle.

53. A method of placing an electrode in muscle or near nerve tissue of a patient, the method comprising:
inserting, under visualization from only a single laparoscopic camera, an introducer needle through a dermal layer and a subcutaneous tissue;
inserting, under visualization from the single laparoscopic camera, an insertion needle through the introducer needle and into a target muscle or near a target nerve tissue, wherein an electrode is loaded into a lumen of the insertion needle; and
deploying the electrode at the target muscle or near the target nerve tissue.

54. The method of claim 53, wherein the target muscle is the diaphragm.
